(19) United States
(12) Patent Application Publication
(10) Pub. No.: US 2020/0385815 A1
(43) Pub. Date: Dec. 10, 2020

(54) Using cfRNA for Diagnosing Minimal Residual Disease

(71) Applicant: NANTOMICS, LLC, Culver City, CA (US)

(72) Inventor: Shahrooz Rabizadeh, Agoura Hills, CA (US)

(21) Appl. No.: 16/768,450

(22) PCT Filed: Dec. 19, 2018
(86) PCT No.: PCT/US2018/066536

§ 371 Date: May 29, 2020

Related U.S. Application Data
(60) Provisional application No. 62/608,321, filed on Dec. 20, 2017.

(51) Int. Cl. C12Q 1/6886

(52) U.S. Cl. CPC C12Q 1/6886; C12Q 2600/106; C12Q 2600/178; C12Q 2600/158

(57) ABSTRACT

cfRNA is used to determine presence or risk of minimal residual disease after treatment of a patient. Most preferably, cfRNA from the patient is analyzed for quantity and/or signatures that are characteristic for the patient's disease.

This application claims priority to our co-pending US provisional application having the Ser. No. 62/608,321, filed Dec. 20, 2017, which is incorporated by reference in its entirety herein.

### FIELD OF THE INVENTION

The field of the invention is analysis of omics data as they relate to cancer, especially as it relates to identification of minimal residual disease using cfRNA.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Information about residual disease after cancer therapy is critical for prediction of therapy success, but also for characterization of remaining cancer cells that are not responsive to the prior therapy. Ideally, residual cancer calls can be analyzed for mutational patterns or other signatures that provide insight to further therapeutic options. However, such analysis is typically limited to scenarios where relatively large numbers of residual cells or tissue are present such that DNA or RNA can be obtained from those residual cells in the blood stream, or where specific mutations are known for a tumor (e.g., bcr/abl fusion) that can be amplified from even a very low number of cells or relatively stable cell free DNAs. However, many patients have idiosyncratic mutations that may even be different among different tumor locations or metastases, which cannot be identified using all or almost all of the known methods. Also, some minimal residual disease may not be associated with any specific mutations on a gene, and rather can be marked with abnormal increase or decrease of specific gene expressions.

Thus, even though some methods for detection of minimal residual disease are known in the art, various disadvantages still remain. Most notably, where minimal residual disease is present with the tumor cells being not readily identifiable, detection of residual cells or tissue is typically not achievable. Therefore, there remains a need for improved methods of analyzing patient samples to detect minimal residual disease.

### SUMMARY OF THE INVENTION

The inventive subject matter is directed to compositions and methods of using cfRNA for diagnosing minimal residual disease (MRD). For example, it is contemplated that before and after surgery, cfRNA associated with a tumor could be identified and tracked to determine if the tumor has indeed been fully removed. Such identification could use idiosyncratic markers, tumor and/or patient-specific signatures, including statistical signatures, and could be compiled across many treatment stages and even across different patients.

In one aspect of the inventive subject matter, the inventor contemplates a method of determining presence of minimal residual disease in a patient. Especially preferred methods include a step of obtaining or identifying sequence information that is specific for at least one expressed gene in a tumor of the patient, wherein the step of obtaining or identifying is performed before treatment of the patient. In a further step, cfRNA is obtained from blood of the patient, typically after treatment of the patient, and the cfRNA is then used to quantify the at least one expressed gene.

Most typically, the step of obtaining sequence information comprises data transfer of sequence data from a database, and/or the step of identifying sequence information comprises omics analysis of the tumor. Moreover, it is generally contemplated that the expressed gene is a cancer-related gene, a cancer-specific gene, a DNA-repair gene, a checkpoint related gene, and/or a gene comprising a sequence encoding a patient- and tumor specific neoepitope. Typically, the sequence information is specific for at least two, or at least five, or at least ten, or at least 50, or at least 100 expressed genes in a tumor of the patient, and/or the treatment of the patient includes chemotherapy, radiation therapy, and/or surgery. It is still further preferred that the cfRNA is substantially devoid of DNA, and/or that the at least one expressed gene is quantified using qPCR. Where desired, a signature may be identified for the at least one expressed gene, and/or the at least one expressed gene may be correlated with a response to the treatment.

In another aspect of the inventive subject matter, the inventor also contemplates a method of determining presence of minimal residual disease in a patient that includes a step of identifying, after treatment of the patient, at least two expressed gene of a treated tumor from cfRNA of the patient, and a further step of correlating presence of minimal residual disease with a threshold quantity and/or pattern of the at least two expressed genes.

In such method, it is generally preferred that the step of identifying the at least two expressed genes further comprises a step of quantifying the cfRNA for the at least two expressed genes. For example, suitable expressed genes will include cancer-related genes, cancer-specific genes, DNA-repair genes, checkpoint related genes, and genes comprising a sequence encoding a patient- and tumor specific neoepitope. As noted above, it is further contemplated that the cfRNA is obtained from blood of the patient, that the cfRNA is substantially devoid of DNA, and/or that the treatment of the patient includes chemotherapy, radiation therapy, and/or surgery.

In further contemplated methods, the threshold quantity may be a detection limit for qPCR (e.g., at least 20% of a measured quantity of at least one of the at least two expressed genes before treatment), and/or the pattern may be a pattern that is characteristic for recurring disease, treatment resistance, and/or immune suppression. Moreover, the pattern may be a pattern from a different patient (which will typically be indicative of minimal residual disease across multiple patients).

Consequently, the inventor also contemplates the use of tumor derived cfRNA of a patient in the determination of minimal residual disease in the patient after treatment of the patient to eradicate the tumor. Most typically, the cfRNA is obtained from blood of the patient. In still further contemplated aspects, the cfRNA includes a sequence that encodes a neoepitope that is tumor specific and patient specific, the cfRNA is further analyzed for at least one of mutations, splice variations, gene copy number, loss of heterozygosity, and epigenetic status, and/or the cfRNA is further analyzed for quantity of the cfRNA. In addition, it should be noted that the cfRNA in all contemplated methods also includes non-coding and regulatory RNA. In particularly contemplated uses, the determination of minimal residual disease includes a determination of a cfRNA quantity, a cfRNA signature, and a cfRNA score, and/or the treatment is at least one of chemotherapy, radiation therapy, and surgery.

Various objects, features, aspects and advantages of the inventive subject matter will become more apparent from the following detailed description of preferred embodiments.

### DETAILED DESCRIPTION

The inventor discovered that minimal residual disease can be detected well in advance of imaging or numerous other diagnostic tests by detecting the presence, a quantity, a score, and/or a pattern of cfRNA in a patient. Most advantageously, such early detection can be performed via a simple blood draw and will not require invasive procedures or imaging processes. Typically, relevant sequences for monitoring are known sequences (e.g., tumor associated or tumor specific antigen encoding RNA) or sequences that were previously identified in the patient tumor and that are specific to the tumor (e.g., tumor and patient specific neoepitopes). Moreover, it should be noted that cfRNA may also include non-coding sequences, and especially regulatory non-coding sequences such as siRNA, shRNA, etc. As will further be appreciated, all of the sequences may be individually relevant to minimal residual disease or may be used collectively to so generate a score or patterns that is indicative to the minimal residual disease (i.e., in most cases primary cancer cells, metastatic cells, or a sub-clonal fraction that is or has become treatment resistant).

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have cancer/minimal residual disease. As used herein, the term “provide” or “providing” refers to and includes any acts of manufacturing, generating, placing, enabling to use, transferring, or making ready to use.

### Cell-Free RNA

The inventors contemplate that tumor cells and/or some immune cells interacting or surrounding the tumor cells release cell free RNA to the patient's bodily fluid, and thus may increase the quantity of the specific cell free RNA in the patient's bodily fluid as compared to a healthy individual. As used herein, the patient's bodily fluid includes, but is not limited to, blood, serum, plasma, mucus, cerebrospinal fluid, ascites fluid, saliva, and urine of the patient. Alternatively, it should be noted that various other bodily fluids are also deemed appropriate so long as cell free RNA is present in such fluids. The patient's bodily fluid may be fresh or preserved/frozen. Appropriate fluids include saliva, ascites fluid, spinal fluid, urine, etc., which may be fresh or preserved/frozen.

The cell free RNA may include any types of RNA that are circulating in the bodily fluid of a person without being enclosed in a cell body or a nucleus. Most typically, the source of the cell free RNA is tumor cells, metastatic cells, or tumor cells dislodged during surgery. However, it is also contemplated that the source of the cell free RNA is an immune cell (e.g., NK cells, T cells, macrophages, etc.). Thus, the cell free RNA can be circulating tumor RNA (ctRNA) and/or circulating free RNA (cfRNA, circulating nucleic acids that do not derive from a tumor). While not wishing to be bound by a particular theory, it is contemplated that release of cell free RNA originating from a tumor cell can be increased when the tumor cell interacts with an immune cell or when the tumor cells undergo cell death (e.g., necrosis, apoptosis, autophagy, etc.). Thus, in some embodiments, the cell free RNA may be enclosed in a vesicular structure (e.g., via exosomal release of cytoplasmic substances) so that it can be protected from nuclease (e.g., RNAase) activity in some type of bodily fluid. Yet, it is also contemplated that in other aspects, the cell free RNA is a naked RNA without being enclosed in any membranous structure, but may be in a stable form by itself or be stabilized via interaction with one or more non-nucleotide molecules (e.g., any RNA binding proteins, etc.).

It is contemplated that the cell free RNA can be any type of RNA which can be released from either cancer cells or immune cell. Thus, the cell free RNA may include mRNA, tRNA, microRNA, small interfering RNA, long non-coding RNA (lncRNA). The cell free RNA may be a fragmented RNA typically with a length of at least 50 base pair (bp), 100 base pair (bp), 200 bp, 500 bp, or 1 kbp. However, it is also contemplated that the cell free RNA is a full length or a fragment of mRNA (e.g., at least 70% of full-length, at least 50% of full length, at least 30% of full length, etc.). While cell free RNA may include any type of RNA encoding any cellular, extracellular proteins or non-protein elements, it is preferred that at least some of cell free RNA encodes one or more cancer-related proteins, or inflammation-related proteins. For example, the cell free mRNA may be full-length or fragments of (or derived from the) cancer related genes including, but not limited to ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDCl73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHAS, EPHA7, EPHB1, ERBB2, ERBB3, ERBB4, EREG, ERG, ERRFI1, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HMGB1, HMGB2, HMGB3, HNF1A, HRAS, HSD3B1, HSP90AA1, IDH1, IDH2, IDO, IGF1R, IGF2, IKBKE, IKZF1, IL7R, INHBA, INPP4B, IRF2, IRF4, IRS2, JAK1, JAK2, JAK3, JUN, MYST3, KDMSA, KDMSC, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRSS8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCB5, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCLS, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGES, XCL1, XCL2, and XCR1. Of course, it should be appreciated that the above genes may be wild type or mutated versions, including missense or nonsense mutations, insertions, deletions, fusions, and/or translocations, all of which may or may not cause formation of full-length mRNA when transcribed.

In other examples, cell free mRNAs are fragments of or those encoding a full length or a fragment of inflammation-related proteins, including, but not limited to, HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, and hTERT, and in yet another example, the cell free mRNA encoded a full length or a fragment of HMGB1.

In yet another example, some cell free mRNAs are fragments of or those encoding a full length or a fragment of DNA repair-related proteins or RNA repair-related proteins. Table 1 provides an exemplary collection of predominant RNA repair genes and their associated repair pathways contemplated herein, but it should be recognized that numerous other genes associated with DNA repair and repair pathways are also expressly contemplated herein, and Tables 2 and 3 illustrate further exemplary genes for analysis and their associated function in DNA repair.

TABLE 1

| Repair mechanism | Predominant DNA Repair genes |
|---|---|
| Base excision | DNA glycosylase, APE1, XRCC1, PNKP, Tdp1, |
| repair (BER) | APTX, DNA polymerase β, FEN1, DNA |
| | polymerase δ or ε, PCNA-RFC, PARP |
| Mismatch repair | MutSα (MSH2-MSH6), Mutsβ (MSH2-MSH3), |
| (MMR) | MutLα (MLH1-PMS2), MutLβ (MLH1-PMS2), |
| | MutLγ (MLH1-MLH3), Exo1, PCNA-RFC |
| Nucleotide | XPC-Rad23B-CEN2, UV-DDB (DDB1-XPE), CSA, |
| excision | CSB, TFIIH, XPB, XPD, XPA, RPA, XPG, |
| repair (NER) | ERCC1- XPF, DNA polymerase δ or ε |
| Homologous | Mre11-Rad50-Nbs1, CtIP, RPA, Rad51, Rad52, |
| recombination | BRCA1, BRCA2, Exo1, BLM-TopIIIα, |
| (HR) | GEN1-Yen1, Slx1-Slx4, Mus81/Eme1 |
| Non-homologous | Ku70-Ku80, DNA-PKc, XRCC4-DNA ligase IV, |
| end-joining | XLF |
| (NHEJ) | |

TABLE 2

| | | Accession |
|---|---|---|
| Gene name (synonyms) | Activity | number |
| Base excision repair (BER) | | |
| | DNA glycosylases: major altered base | |
| | released | |
| UNG | U excision | NM_003362 |
| SMUG1 | U excision | NM_014311 |
| MBD4 | U or T opposite G at CpG sequences | NM_003925 |
| TDG | U, T or ethenoC opposite G | NM_003211 |
| OGG1 | 8-oxoG opposite C | NM_002542 |
| MYH | A opposite 8-oxoG | NM_012222 |
| NTH1 | Ring-saturated or fragmented | NM_002528 |
| | pyrimidines | |
| MPG | 3-meA, ethenoA, hypoxanthine | NM_002434 |
| | Other BER factors | |
| APE1 (HAP1, APEX, REF1) | AP endonuclease | NM_001641 |
| APE2 (APEXL2) | AP endonuclease | NM_014481 |
| LIG3 | Main ligation function | NM_013975 |
| XRCC1 | Main ligation function | NM_006297 |
| | Poly(ADP-ribose) polymerase (PARP) | |
| | enzymes | |
| ADPRT | Protects strand interruptions | NM_001618 |
| ADPRTL2 | PARP-like enzyme | NM_005485 |
| ADPRTL3 | PARP-like enzyme | AF085734 |
| Direct reversal of damage | | |
| MGMT | O6-meG alkyltransferase | NM_002412 |
| Mismatch excision repair | | |
| (MMR) | | |
| MSH2 | Mismatch and loop recognition | NM_000251 |
| MSH3 | Mismatch and loop recognition | NM_002439 |
| MSH6 | Mismatch recognition | NM_000179 |
| MSH4 | MutS homolog specialized for meiosis | NM_002440 |
| MSH5 | MutS homolog specialized for meiosis | NM_002441 |
| PMS1 | Mitochondrial MutL homolog | NM_000534 |
| MLH1 | MutL homolog | NM_000249 |
| PMS2 | MutL homolog | NM_000535 |
| MLH3 | MutL homolog of unknown function | NM_014381 |
| PMS2L3 | MutL homolog of unknown function | D38437 |
| PMS2L4 | MutL homolog of unknown function | D38438 |
| Nucleotide excision repair | | |
| (NER) | | |
| XPC | Binds damaged DNA as complex | NM_004628 |
| RAD23B (HR23B) | Binds damaged DNA as complex | NM_002874 |
| CETN2 | Binds damaged DNA as complex | NM_004344 |
| RAD23A (HR23A) | Substitutes for HR23B | NM_005053 |
| χPA | Binds damaged DNA in preincisioncomplex | NM_000380 |
| RPA1 | Binds DNA in preincision complex | NM_002945 |
| RPA2 | Binds DNA in preincision complex | NM_002946 |
| RPA3 | Binds DNA in preincision complex | NM_002947 |
| TFIIH | Catalyzes unwinding in preincisioncomplex | |
| XPB (ERCC3) | 3′ to 5′ DNA helicase | NM_000122 |
| XPD (ERCC2) | 5′ to 3′ DNA helicase | X52221 |
| GTF2H1 | Core TFIIH subunit p62 | NM_005316 |
| GTF2H2 | Core TFIIH subunit p44 | NM_001515 |
| GTF2H3 | Core TFIIH subunit p34 | NM_001516 |
| GTF2H4 | Core TFIIH subunit p52 | NM_001517 |
| CDK7 | Kinase subunit of TFIIH | NM_001799 |
| CCNH | Kinase subunit of TFIIH | NM_001239 |
| MNAT1 | Kinase subunit of TFIIH | NM_002431 |
| XPG (ERCC5) | 3′ incision | NM_000123 |
| ERCC1 | 5′ incision subunit | NM_001983 |
| XPF (ERCC4) | 5′ incision subunit | NM_005236 |
| LIG1 | DNA joining | NM_000234 |
| NER-related | | |
| CSA (CKN1) | Cockayne syndrome; needed for | NM_000082 |
| | transcription-coupled NER | |
| CSB (ERCC6) | Cockayne syndrome; needed for | NM_000124 |
| | transcription-coupled NER | |
| XAB2 (HCNP) | Cockayne syndrome; needed for | NM_020196 |
| | transcription-coupled NER | |
| DDB1 | Complex defective in XP group E | NM_001923 |
| DDB2 | Mutated in XP group E | NM_000107 |
| MMS19 | Transcription and NER | AW852889 |
| Homologous recombination | | |
| RAD51 | Homologous pairing | NM_002875 |
| RAD51L1 (RAD51B) | Rad51 homolog | U84138 |
| RAD51C | Rad51 homolog | NM_002876 |
| RAD51L3 (RAD51D) | Rad51 homolog | NM_002878 |
| DMC1 | Rad51 homolog, meiosis | NM_007068 |
| XRCC2 | DNA break and cross-link repair | NM_005431 |
| XRCC3 | DNA break and cross-link repair | NM_005432 |
| RAD52 | Accessory factor for recombination | NM_002879 |
| RAD54L | Accessory factor for recombination | NM_003579 |
| RAD54B | Accessory factor for recombination | NM_012415 |
| BRCA1 | Accessory factor for transcription | NM_007295 |
| | and recombination | |
| BRCA2 | Cooperation with RAD51, essential | NM_000059 |
| | function | |
| RAD50 | ATPase in complex with MRE11A, NBS1 | NM_005732 |
| MRE11A | 3′ exonuclease | NM_005590 |
| NBS1 | Mutated in Nijmegen breakage syndrome | NM_002485 |
| Nonhomologous end-joining | | |
| Ku70 (G22P1) | DNA end binding | NM_001469 |
| Ku80 (XRCC5) | DNA end binding | M30938 |
| PRKDC | DNA-dependent protein kinase | NM_006904 |
| | catalytic subunit | |
| LIG4 | Nonhomologous end-joining | NM_002312 |
| XRCC4 | Nonhomologous end-joining | NM_003401 |
| Sanitization of nucleotide pools | | |
| MTH1 (NUDT1) | 8-oxoGTPase | NM_002452 |
| DUT | dUTPase | NM_001948 |
| DNA polymerases (catalytic subunits) | | |
| POLB | BER in nuclear DNA | NM_002690 |
| POLG | BER in mitochondrial DNA | NM_002693 |
| POLD1 | NER and MMR | NM_002691 |
| POLE1 | NER and MMR | NM_006231 |
| PCNA | Sliding clamp for pol delta and pol | NM_002592 |
| | epsilon | |
| REV3L (POLZ) | DNA pol zeta catalytic subunit, | NM_002912 |
| | essential function | |
| REV7 (MAD2L2) | DNA pol zeta subunit | NM_006341 |
| REV1 | dCMP transferase | NM_016316 |
| POLH | XP variant | NM_006502 |
| POLI (RAD30B) | Lesion bypass | NM_007195 |
| POLQ | DNA cross-link repair | NM_006596 |
| DINB1 (POLK) | Lesion bypass | NM_016218 |
| POLL | Meiotic function | NM_013274 |
| POLM | Presumed specialized lymphoid | NM_013284 |
| | function | |
| TRF4-1 | Sister-chromatid cohesion | AF089896 |
| TRF4-2 | Sister-chromatid cohesion | AF089897 |
| Editing and processing nucleases | | |
| FEN1 (DNase IV) | 5′ nuclease | NM_004111 |
| TREX1 (DNase III) | 3′ exonuclease | NM_007248 |
| TREX2 | 3′ exonuclease | NM_007205 |
| EX01 (HEX1) | 5′ exonuclease | NM_003686 |
| SPO11 | endonuclease | NM_012444 |
| Rad6 pathway | | |
| UBE2A (RAD6A) | Ubiquitin-conjugating enzyme | NM_003336 |
| UBE2B (RAD6B) | Ubiquitin-conjugating enzyme | NM_003337 |
| RAD18 | Assists repair or replication of damaged | AB035274 |
| | DNA | |
| UBE2VE (MMS2) | Ubiquitin-conjugating complex | AF049140 |
| UBE2N (UBC13, BTG1) | Ubiquitin-conjugating complex | NM_003348 |
| Genes defective in diseases | | |
| associated with sensitivity to | | |
| DNA damaging agents | | |
| BLM | Bloom syndrome helicase | NM_000057 |
| WRN | Werner syndrome helicase/3′- | NM_000553 |
| | exonuclease | |
| RECQL4 | Rothmund-Thompson syndrome | NM_004260 |
| ATM | Ataxia telangiectasia | NM_000051 |
| Fanconi anemia | | |
| FANCA | Involved in tolerance or repair of DNA | NM_000135 |
| | cross-links | |
| FANCB | Involved in tolerance or repair of DNA | N/A |
| | cross-links | |
| FANCC | Involved in tolerance or repair of DNA | NM_000136 |
| | cross-links | |
| FANCD | Involved in tolerance or repair of DNA | N/A |
| | cross-links | |
| FANCE | Involved in tolerance or repair of DNA | NM_021922 |
| | cross-links | |
| FANCF | Involved in tolerance or repair of DNA | AF181994 |
| | cross-links | |
| FANCG (XRCC9) | Involved in tolerance or repair of DNA | NM_004629 |
| | cross-links | |
| Other identified genes with a | | |
| suspected DNA repair function | | |
| SNM1 (PS02) | DNA cross-link repair | D42045 |
| SNM1B | Related to SNM1 | AL137856 |
| SNM1C | Related to SNM1 | AA315885 |
| RPA4 | Similar to RPA2 | NM_013347 |
| ABH (ALKB) | Resistance to alkylation damage | X91992 |
| PNKP | Converts some DNA breaks to ligatable | NM_007254 |
| | ends | |
| Other conserved DNA | | |
| damage response genes | | |
| ATR | ATM- and PI-3K-like essential kinase | NM_001184 |
| RAD1 (S. pombe) homolog | PCNA-like DNA damage sensor | NM_002853 |
| RAD9 (S. pombe) homolog | PCNA-like DNA damage sensor | NM_004584 |
| HUS1 (S. pombe) homolog | PCNA-like DNA damage sensor | NM_004507 |
| RAD17 (RAD24) | RFC-like DNA damage sensor | NM_002873 |
| TP53BP1 | BRCT protein | NM_005657 |
| CHEK1 | Effector kinase | NM_001274 |
| CHK2 (Rad53) | Effector kinase | NM_007194 |

TABLE 3

| Gene Name | Gene Title | Biological Activity |
|---|---|---|
| RFC2 | replication factor C (activator 1) 2, | DNA replication |
| | 40 kDa | |
| XRCC6 | X-ray repair complementing | DNA ligation /// DNA repair /// double-strand break |
| | defective repair in Chinese hamster | repair via nonhomologous end-joining /// DNA |
| | cells 6 (Ku autoantigen, 70 kDa) | recombination /// positive regulation of |
| | | transcription, DNA-dependent /// double-strand |
| | | break repair via nonhomologous end-joining /// |
| | | response to DNA damage stimulus /// DNA recombination |
| APOBEC | apolipoprotein B mRNA editing | For all of APOBEC1, APOBEC2, APOBEC3A-H, |
| | enzyme, catalytic polypeptide-like | and APOBEC4, cytidine deaminases. |
| POLD2 | polymerase (DNA directed), delta 2, | DNA replication /// DNA replication |
| | regulatory subunit 50 kDa | |
| PCNA | proliferating cell nuclear antigen | regulation of progression through cell cycle /// DNA |
| | | replication /// regulation of DNA replication /// |
| | | DNA repair /// cell proliferation /// |
| | | phosphoinositide-mediated signaling /// DNA replication |
| RPA1 | replication protein A1, 70 kDa | DNA-dependent DNA replication /// DNA repair /// |
| | | DNA recombination /// DNA replication |
| RPA1 | replication protein A1, 70 kDa | DNA-dependent DNA replication /// DNA repair /// |
| | | DNA recombination /// DNA replication |
| RPA2 | replication protein A2, 32 kDa | DNA replication /// DNA-dependent DNA replication |
| ERCC3 | excision repair cross-complementing | DNA topological change /// transcription-coupled |
| | rodent repair deficiency, | nucleotide-excision repair /// transcription /// |
| | complementation group 3 (xeroderma | regulation of transcription, DNA-dependent /// |
| | pigmentosum group B | transcription from RNA polymerase II promoter /// |
| | complementing) | induction of apoptosis /// sensory perception of |
| | | sound /// DNA repair /// nucleotide-excision repair /// |
| | | response to DNA damage stimulus /// DNA repair |
| UNG | uracil-DNA glycosylase | carbohydrate metabolism /// DNA repair /// |
| | | base-excision repair /// response to DNA damage |
| | | stimulus /// DNA repair /// DNA repair |
| ERCC5 | excision repair cross-complementing | transcription-coupled nucleotide-excision |
| | rodent repair deficiency, | repair /// nucleotide-excision repair /// sensory perception |
| | complementation group 5 (xeroderma | of sound /// DNA repair /// response to DNA damage |
| | pigmentosum, complementation | stimulus /// nucleotide-excision repair |
| | group G (Cockayne syndrome)) | |
| MLH1 | mutL homolog 1, colon cancer, | mismatch repair /// cell cycle /// negative regulation |
| | nonpolyposis type 2 (E. coli) | of progression through cell cycle /// DNA |
| | | repair /// mismatch repair /// response to DNA damage |
| | | stimulus |
| LIG1 | ligase I, DNA, ATP-dependent | DNA replication /// DNA repair /// DNA |
| | | recombination /// cell cycle /// morphogenesis /// |
| | | cell division /// DNA repair /// response to DNA |
| | | damage stimulus /// DNA metabolism |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| NBN | nibrin | DNA damage checkpoint /// cell cycle |
| | | checkpoint /// double-strand break repair |
| MSH6 | mutS homolog 6 (E. coli) | mismatch repair /// DNA metabolism /// DNA |
| | | repair /// mismatch repair /// response to DNA damage |
| | | stimulus |
| POLD4 | polymerase (DNA-directed), delta 4 | DNA replication /// DNA replication |
| RFC5 | replication factor C (activator 1) 5, | DNA replication /// DNA repair /// DNA replication |
| | 36.5 kDa | |
| RFC5 | replication factor C (activator 1) 5, | DNA replication /// DNA repair /// DNA replication |
| | 36.5 kDa | |
| DDB2 /// | damage-specific DNA binding | nucleotide-excision repair /// regulation of |
| LHX3 | protein 2, 48 kDa /// LIM homeobox 3 | transcription, DNA-dependent /// organ |
| | | morphogenesis /// DNA repair /// response to DNA |
| | | damage stimulus /// DNA repair /// transcription /// regulation |
| | | of transcription |
| POLD1 | polymerase (DNA directed), delta 1, | DNA replication /// DNA repair /// response to UV /// DNA |
| | catalytic subunit 125 kDa | replication |
| FANCG | Fanconi anemia, complementation | cell cycle checkpoint /// DNA repair /// DNA |
| | group G | repair /// response to DNA damage stimulus /// regulation |
| | | of progression through cell cycle |
| POLB | polymerase (DNA directed), beta | DNA-dependent DNA replication /// DNA repair /// DNA |
| | | replication /// DNA repair /// response to DNA |
| | | damage stimulus |
| XRCC1 | X-ray repair complementing | single strand break repair |
| | defective repair in Chinese hamster | |
| | cells 1 | |
| MPG | N-methylpurine-DNA glycosylase | base-excision repair /// DNA dealkylation /// DNA |
| | | repair /// base-excision repair /// response to DNA |
| | | damage stimulus |
| RFC2 | replication factor C (activator 1) 2, | DNA replication |
| | 40 kDa | |
| ERCC1 | excision repair cross-complementing | nucleotide-excision repair /// morphogenesis /// |
| | rodent repair deficiency, | nucleotide-excision repair /// DNA repair /// |
| | complementation group 1 (includes | response to DNA damage stimulus |
| | overlapping antisense sequence) | |
| TDG | thymine-DNA glycosylase | carbohydrate metabolism /// base-excision repair /// DNA |
| | | repair /// response to DNA damage stimulus |
| TDG | thymine-DNA glycosylase | carbohydrate metabolism /// base-excision repair /// DNA |
| | | repair /// response to DNA damage stimulus |
| FANCA | Fanconi anemia, complementation | DNA repair /// protein complex assembly /// DNA |
| | group A /// Fanconi anemia, | repair /// response to DNA damage stimulus |
| | complementation group A | |
| RFC4 | replication factor C (activator 1) 4, | DNA replication /// DNA strand elongation /// DNA |
| | 37 kDa | repair /// phosphoinositide-mediated signaling /// DNA |
| | | replication |
| RFC3 | replication factor C (activator 1) 3, | DNA replication /// DNA strand elongation |
| | 38 kDa | |
| RFC3 | replication factor C (activator 1) 3, | DNA replication /// DNA strand elongation |
| | 38 kDa | |
| APEX2 | APEX nuclease | DNA repair /// response to DNA damage stimulus |
| | (apurinic/apyrimidinic endonuclease) | |
| | 2 | |
| RAD1 | RAD1 homolog (S. pombe) | DNA repair /// cell cycle checkpoint /// cell cycle |
| | | checkpoint /// DNA damage checkpoint /// DNA |
| | | repair /// response to DNA damage stimulus /// meiotic |
| | | prophase I |
| RAD1 | RAD1 homolog (S. pombe) | DNA repair /// cell cycle checkpoint /// cell cycle |
| | | checkpoint /// DNA damage checkpoint /// DNA |
| | | repair /// response to DNA damage stimulus /// meiotic |
| | | prophase I |
| BRCA1 | breast cancer 1, early onset | regulation of transcription from RNA polymerase II |
| | | promoter /// regulation of transcription from RNA |
| | | polymerase III promoter /// DNA damage response, |
| | | signal transduction by p53 class mediator resulting |
| | | in transcription of p21 class mediator /// cell |
| | | cycle /// protein ubiquitination /// androgen receptor |
| | | signaling pathway /// regulation of cell |
| | | proliferation /// regulation of apoptosis /// positive |
| | | regulation of DNA repair /// negative regulation of |
| | | progression through cell cycle /// positive regulation of |
| | | transcription, DNA-dependent /// negative |
| | | regulation of centriole replication /// DNA damage |
| | | response, signal transduction resulting in induction |
| | | of apoptosis /// DNA repair /// response to DNA |
| | | damage stimulus /// protein ubiquitination /// DNA |
| | | repair /// regulation of DNA repair /// apoptosis /// response |
| | | to DNA damage stimulus |
| EXO1 | exonuclease 1 | DNA repair /// DNA repair /// mismatch repair /// DNA |
| | | recombination |
| FEN1 | flap structure-specific endonuclease 1 | DNA replication /// double-strand break repair /// UV |
| | | protection /// phosphoinositide-mediated |
| | | signaling /// DNA repair /// DNA replication /// DNA |
| | | repair /// DNA repair |
| FEN1 | flap structure-specific endonuclease 1 | DNA replication /// double-strand break repair /// UV |
| | | protection /// phosphoinositide-mediated |
| | | signaling /// DNA repair /// DNA replication /// DNA |
| | | repair /// DNA repair |
| MLH3 | mutL homolog 3 (E. coli) | mismatch repair /// meiotic recombination /// DNA |
| | | repair /// mismatch repair /// response to DNA |
| | | damage stimulus /// mismatch repair |
| MGMT | O-6-methylguanine-DNA | DNA ligation /// DNA repair /// response to DNA |
| | methyltransferase | damage stimulus |
| RAD51 | RAD51 homolog (RecA homolog, | double-strand break repair via homologous |
| | E. coli) (S. cerevisiae) | recombination /// DNA unwinding during |
| | | replication /// DNA repair /// mitotic |
| | | recombination /// meiosis /// meiotic |
| | | recombination /// positive |
| | | regulation of DNA ligation /// protein |
| | | homooligomerization /// response to DNA damage |
| | | stimulus /// DNA metabolism /// DNA repair /// response to |
| | | DNA damage stimulus /// DNA repair /// DNA |
| | | recombination /// meiotic recombination /// double-strand |
| | | break repair via homologous |
| | | recombination /// DNA unwinding during |
| | | replication |
| RAD51 | RAD51 homolog (RecA homolog, | double-strand break repair via homologous |
| | E. coli) (S. cerevisiae) | recombination /// DNA unwinding during |
| | | replication /// DNA repair /// mitotic |
| | | recombination /// meiosis /// meiotic |
| | | recombination /// positive |
| | | regulation of DNA ligation /// protein |
| | | homooligomerization /// response to DNA damage |
| | | stimulus /// DNA metabolism /// DNA repair /// response to |
| | | DNA damage stimulus /// DNA repair /// DNA |
| | | recombination /// meiotic recombination /// double-strand |
| | | break repair via homologous recombination /// DNA |
| | | unwinding during replication |
| XRCC4 | X-ray repair complementing | DNA repair /// double-strand break repair /// DNA |
| | defective repair in Chinese hamster | recombination /// DNA recombination /// response |
| | cells 4 | to DNA damage stimulus |
| XRCC4 | X-ray repair complementing | DNA repair /// double-strand break repair /// DNA |
| | defective repair in Chinese hamster | recombination /// DNA recombination /// response |
| | cells 4 | to DNA damage stimulus |
| RECQL | RecQ protein-like (DNA helicase | DNA repair /// DNA metabolism |
| | Q1-like) | |
| ERCC8 | excision repair cross-complementing | DNA repair /// transcription /// regulation of |
| | rodent repair deficiency, | transcription, DNA-dependent /// sensory perception |
| | complementation group 8 | of sound /// transcription-coupled nucleotide-excision |
| | | repair |
| FANCC | Fanconi anemia, complementation | DNA repair /// DNA repair /// protein complex |
| | group C | assembly /// response to DNA damage stimulus |
| OGG1 | 8-oxoguanine DNA glycosylase | carbohydrate metabolism /// base-excision repair /// DNA |
| | | repair /// base-excision repair /// response to DNA damage |
| | | stimulus /// DNA repair |
| MRE11A | MRE11 meiotic recombination 11 | regulation of mitotic recombination /// double-strand |
| | homolog A (S. cerevisiae) | break repair via nonhomologous |
| | | end-joining /// telomerase-dependent telomere |
| | | maintenance /// meiosis /// meiotic recombination /// DNA |
| | | metabolism /// DNA repair /// double-strand break |
| | | repair /// response to DNA damage stimulus /// DNA |
| | | repair /// double-strand break repair /// DNA |
| | | recombination |
| RAD52 | RAD52 homolog (S. cerevisiae) | double-strand break repair /// mitotic |
| | | recombination /// meiotic recombination /// DNA |
| | | repair /// DNA recombination /// response to DNA damage |
| | | stimulus |
| WRN | Werner syndrome | DNA metabolism /// aging |
| XPA | xeroderma pigmentosum, | nucleotide-excision repair /// DNA repair /// response to |
| | complementation group A | DNA damage stimulus /// DNA |
| | | repair /// nucleotide-excision repair |
| BLM | Bloom syndrome | DNA replication /// DNA repair /// DNA |
| | | recombination /// antimicrobial humoral response |
| | | (sensu Vertebrata) /// DNA metabolism /// DNA |
| | | replication |
| OGG1 | 8-oxoguanine DNA glycosylase | carbohydrate metabolism /// base-excision repair /// DNA |
| | | repair /// base-excision repair /// response to |
| | | DNA damage stimulus /// DNA repair |
| MSH3 | mutS homolog 3 (E. coli) | mismatch repair /// DNA metabolism /// DNA |
| | | repair /// mismatch repair /// response to DNA damage |
| | | stimulus |
| POLE2 | polymerase (DNA directed), epsilon | DNA replication /// DNA repair /// DNA replication |
| | 2 (p59 subunit) | |
| RAD51C | RAD51 homolog C (S. cerevisiae) | DNA repair /// DNA recombination /// DNA |
| | | metabolism /// DNA repair /// DNA recombination /// |
| | | response to DNA damage stimulus |
| LIG4 | ligase IV, DNA, ATP-dependent | single strand break repair /// DNA replication /// DNA |
| | | recombination /// cell cycle /// cell division /// DNA |
| | | repair /// response to DNA damage stimulus |
| ERCC6 | excision repair cross-complementing | DNA repair /// transcription /// regulation of |
| | rodent repair deficiency, | transcription, DNA-dependent /// transcription from |
| | complementation group 6 | RNA polymerase II promoter /// sensory perception |
| | | of sound |
| LIG3 | ligase III, DNA, ATP-dependent | DNA replication /// DNA repair /// cell cycle /// meiotic |
| | | recombination /// spermatogenesis /// cell |
| | | division /// DNA repair /// DNA recombination /// response to |
| | | DNA damage stimulus |
| RAD17 | RAD17 homolog (S. pombe) | DNA replication /// DNA repair /// cell cycle /// response to |
| | | DNA damage stimulus |
| XRCC2 | X-ray repair complementing | DNA repair /// DNA recombination /// meiosis /// DNA |
| | defective repair in Chinese hamster | metabolism /// DNA repair /// response to |
| | cells 2 | DNA damage stimulus |
| MUTYH | mutY homolog (E. coli) | carbohydrate metabolism /// base-excision |
| | | repair /// mismatch repair /// cell cycle /// negative regulation |
| | | of progression through cell cycle /// DNA |
| | | repair /// response to DNA damage stimulus /// DNA repair |
| RFC1 | replication factor C (activator 1) 1, | DNA-dependent DNA replication /// transcription /// |
| | 145 kDa /// replication factor C | regulation of transcription, DNA-dependent /// |
| | (activator 1) 1, 145 kDa | telomerase-dependent telomere maintenance /// |
| | | DNA replication /// DNA repair |
| RFC1 | replication factor C (activator 1) 1, | DNA-dependent DNA replication /// transcription /// |
| | 145 kDa | regulation of transcription, DNA-dependent /// |
| | | telomerase-dependent telomere maintenance /// |
| | | DNA replication /// DNA repair |
| BRCA2 | breast cancer 2, early onset | regulation of progression through cell |
| | | cycle /// double-strand break repair via homologous |
| | | recombination /// DNA repair /// establishment |
| | | and/or maintenance of chromatin architecture /// |
| | | chromatin remodeling /// regulation of S phase of |
| | | mitotic cell cycle /// mitotic checkpoint /// |
| | | regulation of transcription /// response to DNA |
| | | damage stimulus |
| RAD50 | RAD50 homolog (S. cerevisiae) | regulation of mitotic recombination /// |
| | | double-strand break repair /// telomerase-dependent |
| | | telomere maintenance /// cell cycle /// meiosis /// meiotic |
| | | recombination /// chromosome organization |
| | | and biogenesis /// telomere maintenance /// DNA |
| | | repair /// response to DNA damage stimulus /// DNA |
| | | repair /// DNA recombination |
| DDB1 | damage-specific DNA binding | nucleotide-excision repair /// ubiquitin cycle /// DNA |
| | protein 1, 127 kDa | repair /// response to DNA damage stimulus /// DNA repair |
| XRCC5 | X-ray repair complementing | double-strand break repair via nonhomologous |
| | defective repair in Chinese hamster | end-joining /// DNA recombination /// DNA repair /// DNA |
| | cells 5 (double-strand-break | recombination /// response to DNA damage |
| | rejoining; Ku autoantigen, 80 kDa) | stimulus /// double-strand break repair |
| XRCC5 | X-ray repair complementing | double-strand break repair via nonhomologous |
| | defective repair in Chinese hamster | end-joining /// DNA recombination /// DNA repair /// DNA |
| | cells 5 (double-strand-break | recombination /// response to DNA damage |
| | rejoining; Ku autoantigen, 80 kDa) | stimulus /// double-strand break repair |
| PARP1 | poly (ADP-ribose) polymerase | DNA repair /// transcription from RNA polymerase II |
| | family, member 1 | promoter /// protein amino acid ADP-ribosylation /// DNA |
| | | metabolism /// DNA repair /// protein amino acid |
| | | ADP-ribosylation /// response to DNA damage stimulus |
| POLE3 | polymerase (DNA directed), epsilon | DNA replication |
| | 3 (p17 subunit) | |
| RFC1 | replication factor C (activator 1) 1, | DNA-dependent DNA |
| | 145 kDa | replication /// transcription /// regulation of transcription, |
| | | DNA-dependent /// telomerase-dependent telomere |
| | | maintenance /// DNA replication /// DNA repair |
| RAD50 | RAD50 homolog (S. cerevisiae) | regulation of mitotic recombination /// double- |
| | | strand break repair /// telomerase-dependent |
| | | telomere maintenance /// cell cycle /// meiosis /// meiotic |
| | | recombination /// chromosome organization |
| | | and biogenesis /// telomere maintenance /// DNA |
| | | repair /// response to DNA damage stimulus /// DNA |
| | | repair /// DNA recombination |
| XPC | xeroderma pigmentosum, | nucleotide-excision repair /// DNA |
| | complementation group C | repair /// nucleotide-excision repair /// response to DNA |
| | | damage stimulus /// DNA repair |
| MSH2 | mutS homolog 2, colon cancer, | mismatch repair /// postreplication repair /// cell |
| | nonpolyposis type 1 (E. coli) | cycle /// negative regulation of progression through |
| | | cell cycle /// DNA metabolism /// DNA repair /// mismatch |
| | | repair /// response to DNA damage stimulus /// DNA repair |
| RPA3 | replication protein A3, 14 kDa | DNA replication /// DNA repair /// DNA replication |
| MBD4 | methyl-CpG binding domain protein | base-excision repair /// DNA repair /// response to |
| | 4 | DNA damage stimulus /// DNA repair |
| MBD4 | methyl-CpG binding domain protein | base-excision repair /// DNA repair /// response to |
| | 4 | DNA damage stimulus /// DNA repair |
| NTHL1 | nth endonuclease III-like 1 (E. coli) | carbohydrate metabolism /// base-excision |
| | | repair /// nucleotide-excision repair, DNA incision, |
| | | 5′-to lesion /// DNA repair /// response to DNA |
| | | damage stimulus |
| PMS2 /// | PMS2 postmeiotic segregation | mismatch repair /// cell cycle /// negative regulation |
| PMS2CL | increased 2 (S. cerevisiae) /// | of progression through cell cycle /// DNA |
| | PMS2-C terminal-like | repair /// mismatch repair /// response to DNA damage |
| | | stimulus /// mismatch repair |
| RAD51C | RAD51 homolog C (S. cerevisiae) | DNA repair /// DNA recombination /// DNA |
| | | metabolism /// DNA repair /// DNA |
| | | recombination /// response to DNA damage stimulus |
| UNG2 | uracil-DNA glycosylase 2 | regulation of progression through cell |
| | | cycle /// carbohydrate metabolism /// base-excision |
| | | repair /// DNA repair /// response to DNA damage stimulus |
| APEX1 | APEX nuclease (multifunctional | base-excision repair /// transcription from RNA |
| | DNA repair enzyme) 1 | polymerase II promoter /// regulation of DNA |
| | | binding /// DNA repair /// response to DNA damage |
| | | stimulus |
| ERCC4 | excision repair cross-complementing | nucleotide-excision repair /// nucleotide-excision |
| | rodent repair deficiency, | repair /// DNA metabolism /// DNA repair /// response to |
| | complementation group 4 | DNA damage stimulus |
| RAD1 | RAD1 homolog (S. pombe) | DNA repair /// cell cycle checkpoint /// cell cycle |
| | | checkpoint /// DNA damage checkpoint /// DNA |
| | | repair /// response to DNA damage stimulus /// meiotic |
| | | prophase I |
| RECQL5 | RecQ protein-like 5 | DNA repair /// DNA metabolism /// DNA metabolism |
| MSH5 | mutS homolog 5 (E. coli) | DNA metabolism /// mismatch repair /// mismatch |
| | | repair /// meiosis /// meiotic recombination /// meiotic |
| | | prophase II /// meiosis |
| RECQL | RecQ protein-like (DNA helicase | DNA repair /// DNA metabolism |
| | Q1-like) | |
| RAD52 | RAD52 homolog (S. cerevisiae) | double-strand break repair /// mitotic |
| | | recombination /// meiotic recombination /// DNA |
| | | repair /// DNA recombination /// response to DNA damage |
| | | stimulus |
| XRCC4 | X-ray repair complementing | DNA repair /// double-strand break repair /// DNA |
| | defective repair in Chinese hamster | recombination /// DNA recombination /// response |
| | cells 4 | to DNA damage stimulus |
| XRCC4 | X-ray repair complementing | DNA repair /// double-strand break repair /// DNA |
| | defective repair in Chinese hamster | recombination /// DNA recombination /// response |
| | cells 4 | to DNA damage stimulus |
| RAD17 | RAD17 homolog (S. pombe) | DNA replication /// DNA repair /// cell cycle /// response to |
| | | DNA damage stimulus |
| MSH3 | mutS homolog 3 (E. coli) | mismatch repair /// DNA metabolism /// DNA |
| | | repair /// mismatch repair /// response to DNA damage |
| | | stimulus |
| MRE11A | MRE11 meiotic recombination 11 | regulation of mitotic recombination /// double- |
| | homolog A (S. cerevisiae) | strand break repair via nonhomologous |
| | | end-joining /// telomerase-dependent telomere |
| | | maintenance /// meiosis /// meiotic recombination /// DNA |
| | | metabolism /// DNA repair /// double-strand break |
| | | repair /// response to DNA damage stimulus /// DNA |
| | | repair /// double-strand break repair /// DNA |
| | | recombination |
| MSH6 | mutS homolog 6 (E. coli) | mismatch repair /// DNA metabolism /// DNA |
| | | repair /// mismatch repair /// response to DNA damage |
| | | stimulus |
| MSH6 | mutS homolog 6 (E. coli) | mismatch repair /// DNA metabolism /// DNA |
| | | repair /// mismatch repair /// response to DNA damage |
| | | stimulus |
| RECQL5 | RecQ protein-like 5 | DNA repair /// DNA metabolism /// DNA metabolism |
| BRCA1 | breast cancer 1, early onset | regulation of transcription from RNA polymerase II |
| | | promoter /// regulation of transcription from RNA |
| | | polymerase III promoter /// DNA damage response, |
| | | signal transduction by p53 class mediator resulting |
| | | in transcription of p21 class mediator /// cell |
| | | cycle /// protein ubiquitination /// androgen receptor |
| | | signaling pathway /// regulation of cell |
| | | proliferation /// regulation of apoptosis /// positive |
| | | regulation of DNA repair /// negative regulation of |
| | | progression through cell cycle /// positive regulation of |
| | | transcription, DNA-dependent /// negative |
| | | regulation of centriole replication /// DNA damage |
| | | response, signal transduction resulting in induction |
| | | of apoptosis /// DNA repair /// response to DNA |
| | | damage stimulus /// protein ubiquitination /// DNA |
| | | repair /// regulation of DNA repair /// apoptosis /// response |
| | | to DNA damage stimulus |
| RAD52 | RAD52 homolog (S. cerevisiae) | double-strand break repair /// mitotic |
| | | recombination /// meiotic recombination /// DNA |
| | | repair /// DNA recombination /// response to DNA damage |
| | | stimulus |
| POLD3 | polymerase (DNA-directed), delta 3, | DNA synthesis during DNA repair /// mismatch |
| | accessory subunit | repair /// DNA replication |
| MSH5 | mutS homolog 5 (E. coli) | DNA metabolism /// mismatch repair /// mismatch |
| | | repair /// meiosis /// meiotic recombination /// meiotic |
| | | prophase II /// meiosis |
| ERCC2 | excision repair cross-complementing | transcription-coupled nucleotide-excision repair /// |
| | rodent repair deficiency, | transcription /// regulation of transcription, |
| | complementation group 2 (xeroderma | DNA-dependent /// transcription from RNA polymerase II |
| | pigmentosum D) | promoter /// induction of apoptosis /// sensory |
| | | perception of sound /// nucleobase, nucleoside, |
| | | nucleotide and nucleic acid metabolism /// |
| | | nucleotide-excision repair |
| RECQL4 | RecQ protein-like 4 | DNA repair /// development /// DNA metabolism |
| PMS1 | PMS1 postmeiotic segregation | mismatch repair /// regulation of transcription, |
| | increased 1 (S. cerevisiae) | DNA-dependent /// cell cycle /// negative regulation |
| | | of progression through cell cycle /// mismatch |
| | | repair /// DNA repair /// response to DNA damage |
| | | stimulus |
| ZFP276 | zinc finger protein 276 homolog | transcription /// regulation of transcription, |
| | (mouse) | DNA-dependent |
| MBD4 | methyl-CpG binding domain protein | base-excision repair /// DNA repair /// response to |
| | 4 | DNA damage stimulus /// DNA repair |
| MBD4 | methyl-CpG binding domain protein | base-excision repair /// DNA repair /// response to |
| | 4 | DNA damage stimulus /// DNA repair |
| MLH3 | mutL homolog 3 (E. coli) | mismatch repair /// meiotic recombination /// DNA |
| | | repair /// mismatch repair /// response to DNA |
| | | damage stimulus /// mismatch repair |
| FANCA | Fanconi anemia, complementation | DNA repair /// protein complex assembly /// DNA |
| | group A | repair /// response to DNA damage stimulus |
| POLE | polymerase (DNA directed), epsilon | DNA replication /// DNA repair /// DNA |
| | | replication /// response to DNA damage stimulus |
| XRCC3 | X-ray repair complementing | DNA repair /// DNA recombination /// DNA |
| | defective repair in Chinese hamster | metabolism /// DNA repair /// DNA |
| | cells 3 | recombination /// response to DNA damage |
| | | stimulus /// response to DNA damage stimulus |
| MLH3 | mutL homolog 3 (E. coli) | mismatch repair /// meiotic recombination /// DNA |
| | | repair /// mismatch repair /// response to DNA |
| | | damage stimulus /// mismatch repair |
| NBN | nibrin | DNA damage checkpoint /// cell cycle |
| | | checkpoint /// double-strand break repair |
| SMUG1 | single-strand selective | carbohydrate metabolism /// DNA repair /// response |
| | monofunctional uracil DNA | to DNA damage stimulus |
| | glycosylase | |
| FANCF | Fanconi anemia, complementation | DNA repair /// response to DNA damage stimulus |
| | group F | |
| NEIL1 | nei endonuclease VIII-like 1 (E. coli) | carbohydrate metabolism /// DNA repair /// response |
| | | to DNA damage stimulus |
| FANCE | Fanconi anemia, complementation | DNA repair /// response to DNA damage stimulus |
| | group E | |
| MSH5 | mutS homolog 5 (E. coli) | DNA metabolism /// mismatch repair /// mismatch |
| | | repair /// meiosis /// meiotic recombination /// meiotic |
| | | prophase II /// meiosis |
| RECQL5 | RecQ protein-like 5 | DNA repair /// DNA metabolism /// DNA metabolism |

In still another example, some cell free RNAs are derived from specific genes that are known or implicated to contribute to the development or progress of various types of minimal residual diseases (e.g., minimal residual disease in childhood acute lymphoblastic leukemia, etc.). Those genes may include one or more of apoptosis-related genes (e.g., caspase-8), BCL2, BECN1, CBFB, IKZF1, PAX5, SH2B3, TOX, BHLHE40, BIRC5, C2ORF27, C7ORF25, CC2D1A, CD8A, CDK16, CES2, CHAT, FAM204A, ICOS, RYBP, CLIP3, ZHX2, BMP8A, MPL, MYH11, TCL6, SLC7A6, ANKRD40, ATF7IP, ATG4B, C150RF63, CEPT1, DNAJC13, DOCK2, FAM48A, FTO, GUCY1A3, CTDSPL, FGF17, HIST1H2AB, IL8, ITGB3, KDM3A, MYL6, NPDC1, ST8SIA3, and TSPYL2, etc.

In still another example, some cell free RNAs are fragments of or those encoding a full length or a fragment of a gene not associated with a disease (e.g., housekeeping genes), including, but not limited to, those related to transcription factors (e.g., ATF1, ATF2, ATF4, ATF6, ATF7, ATFIP, BTF3, E2F4, ERH, HMGB1, ILF2, IER2, JUND, TCEB2, etc.), repressors (e.g., PUF60), RNA splicing (e.g., BAT1, HNRPD, HNRPK, PABPN1, SRSF3, etc.), translation factors (EIF1, EIF1AD, EIF1B, EIF2A, EIF2AK1, EIF2AK3, EIF2AK4, EIF2B2, EIF2B3, EIF2B4, EIF2S2, EIF3A, etc.), tRNA synthetases (e.g., AARS, CARS, DARS, FARS, GARS, HARS, IARS, KARS, MARS, etc.), RNA binding protein (e.g., ELAVL1, etc.), ribosomal proteins (e.g., RPL5, RPL8, RPL9, RPL10, RPL11, RPL14, RPL25, etc.), mitochondrial ribosomal proteins (e.g., MRPL9, MRPL1, MRPL10, MRPL11, MRPL12, MRPL13, MRPL14, etc.), RNA polymerase (e.g., POLR1C, POLR1D, POLR1E, POLR2A, POLR2B, POLR2C, POLR2D, POLR3C, etc.), protein processing (e.g., PPID, PPI3, PPIF, CANX, CAPN1, NACA, PFDN2, SNX2, SS41, SUMO1, etc.), heat shock proteins (e.g., HSPA4, HSPA5, HSBP1, etc.), histone (e.g., HIST1HSBC, H1FX, etc.), cell cycle (e.g., ARHGAP35, RAB10, RAB11A, CCNY, CCNL, PPP1CA, RAD1, RAD17, etc.), carbohydrate metabolism (e.g., ALDOA, GSK3A, PGK1, PGAM5, etc.), lipid metabolism (e.g., HADHA), citric acid cycle (e.g., SDHA, SDHB, etc.), amino acid metabolism (e.g., COMT, etc.), NADH dehydrogenase (e.g., NDUFA2, etc.), cytochrome c oxidase (e.g., COX5B, COX8, COX11, etc.), ATPase (e.g. ATP2C1, ATP5F1, etc.), lysosome (e.g., CTSD, CSTB, LAMP1, etc.), proteasome (e.g., PSMA1, UBA1, etc.), cytoskeletal proteins (e.g., ANXA6, ARPC2, etc.), and organelle synthesis (e.g., BLOC1S1, AP2A1, etc.).

In still another example, some cell free RNAs are fragments of or those encoding a full length or a fragment of a neoepitope specific to the tumor. With respect to neoepitope, it should be appreciated that neoepitopes can be characterized as random mutations in tumor cells that create unique and tumor specific antigens. Therefore, high-throughput genome sequencing should allow for rapid and specific identification of patient specific neoepitopes where the analysis also considers matched normal tissue of the same patient. In some embodiments, neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous comparison of the so obtained omics information. While not limiting to the inventive subject matter, it is typically preferred that the data are patient matched tumor data (e.g., tumor versus same patient normal), and that the data format is in SAM, BAM, GAR, or VCF format. However, non-matched or matched versus other reference (e.g., prior same patient normal or prior same patient tumor, or homo statisticus) are also deemed suitable for use herein. Therefore, the omics data may be ‘fresh’ omics data or omics data that were obtained from a prior procedure (or even different patient). However, and especially where genomics ctDNA is analyzed, the neoepitope-coding sequence need not necessarily be expressed.

In particularly preferred aspects, the nucleic acid encoding a neoepitope may encode a neoepitope that is also a suitable target for immune therapy. Therefore, neoepitopes can then be further filtered for a match to the patient's HLA type to thereby increase likelihood of antigen presentation of the neoepitope. Most preferably, and as further discussed below, such matching can be done in silico. Most typically, the patient-specific epitopes are unique to the patient, but may also in at least some cases include tumor type-specific neoepitopes (e.g., Her-2, PSA, brachyury) or cancer-associated neoepitopes (e.g., CEA, MUC-1, CYPB1).

It is contemplated that cell free RNA may present in modified forms or different isoforms. For example, the cell free mRNA may be present in a plurality of isoforms (e.g., splicing variants, etc.) that may be associated with different cell types and/or location. Preferably, different isoforms of mRNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, mRNA encoding HMGB1 are present in 18 different alternative splicing variants and 2 unspliced forms. Those isoforms are expected to express in different tissues/locations of the patient's body (e.g., isoform A is specific to prostate, isoform B is specific to brain, isoform C is specific to spleen, etc.). Thus, in these embodiments, identifying the isoforms of cell free mRNA in the patient's bodily fluid can provide information on the origin (e.g., cell type, tissue type, etc.) of the cell free mRNA.

The inventors contemplate that the quantities and/or isoforms (or subtypes) or regulatory noncoding RNA (e.g., microRNA, small interfering RNA, long non-coding RNA (lncRNA)) can vary and fluctuate by presence of a tumor or immune response against the tumor. Without wishing to be bound by any specific theory, varied expression of regulatory noncoding RNA in a cancer patient's bodily fluid may due to genetic modification of the cancer cell (e.g., deletion, translocation of parts of a chromosome, etc.), and/or inflammations at the cancer tissue by immune system (e.g., regulation of miR-29 family by activation of interferon signaling and/or virus infection, etc.). Thus, in some embodiments, the cell free RNA can be a regulatory noncoding RNA that modulates expression (e.g., downregulates, silences, etc.) of mRNA encoding a cancer-related protein or an inflammation-related protein (e.g., HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, hTERT, etc.).

It is also contemplated that some cell free regulatory noncoding RNA may be present in a plurality of isoforms or members (e.g., members of miR-29 family, etc.) that may be associated with different cell types and/or location. Preferably, different isoforms or members of regulatory noncoding RNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, higher expression level of miR-155 in the bodily fluid can be associated with the presence of breast tumor, and the reduced expression level of miR-155 can be associated with reduced size of breast tumor. Thus, in these embodiments, identifying the isoforms of cell free regulatory noncoding RNA in the patient's bodily fluid can provide information on the origin (e.g., cell type, tissue type, etc.) of the cell free regulatory noncoding RNA. Lastly, while the above discussed RNA, it should be appreciated that contemplated systems and methods will also include analyses that determine and quantitate cfDNA.

### Isolation and Amplification of Cell Free DNA/RNA

Any suitable methods to isolate and amplify cell free DNA/RNA are contemplated. Most typically, cell free DNA/RNA is isolated from a bodily fluid (e.g., whole blood) that is processed under a suitable conditions, including a condition that stabilizes cell free RNA. Preferably, both cell free DNA and RNA are isolated simultaneously from the same badge of the patient's bodily fluid. Yet, it is also contemplated that the bodily fluid sample can be divided into two or more smaller samples from which DNA or RNA can be isolated separately. Once separated from the non-nucleic acid components, cell free RNA are then quantified, preferably using real time, quantitative PCR or real time, quantitative RT-PCR. Therefore, and as described in more detail below, contemplated cfRNA will be substantially free from (cf)DNA.

The bodily fluid of the patient can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the cell free DNA/RNA data with the prognosis of the cancer. In some embodiments, the bodily fluid of the patient can be obtained from a patient before and after the cancer treatment (e.g., chemotherapy, radiotherapy, drug treatment, cancer immunotherapy, etc.). Such treatment sampling is especially relevant where the cfRNA is genuine to the tumor (or metastasis) of the patient as these mutations are idiosyncratic and particularly advantageous. Moreover, where sampling is done before treatment, changes in quantities, patterns, or signatures may reflect clonal changes or be indicative of likely treatment outcome. However, where the tumor has one or more common or otherwise known mutations (e.g., KRAS12D, bcr/abl, etc.), sampling may be performed only after treatment started or once treatment concluded.

While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.). Of course, it should be noted that the appropriate sampling time, period, and/or iterations may vary, and the PHOSITA will be readily apprised of suitable protocols.

Additionally or alternatively, the bodily fluid of a healthy individual can be obtained to compare the sequence/modification of cell free DNA, and/or quantity/subtype expression of cell free RNA. As used herein, a healthy individual refers an individual without a tumor. Preferably, the healthy individual can be chosen among group of people shares characteristics with the patient (e.g., age, gender, ethnicity, diet, living environment, family history, etc.). Likewise, bodily fluids of individuals diagnosed with the same disease and/or subjected to the same treatment regimen may be collected for reference and identification of minimal residual disease using statistical protocols and/or machine learning algorithms.

Any suitable methods for isolating cell free DNA/RNA are contemplated. For example, in one exemplary method of DNA isolation, specimens were accepted as 10 ml of whole blood drawn into a test tube. Cell free DNA can be isolated from other from mono-nucleosomal and di-nucleosomal complexes using magnetic beads that can separate out cell free DNA at a size between 100-300 bps. For another example, in one exemplary method of RNA isolation, specimens were accepted as 10 ml of whole blood drawn into cell-free RNA BCT® tubes or cell-free DNA BCT® tubes containing RNA stabilizers, respectively. Advantageously, cell free RNA is stable in whole blood in the cell-free RNA BCT tubes for seven days while cell free RNA is stable in whole blood in the cell-free DNA BCT Tubes for fourteen days, allowing time for shipping of patient samples from world-wide locations without the degradation of cell free RNA. Moreover, it is generally preferred that the cell free RNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cell free RNA in a collected tissue other than blood for at 2 days, more preferably at least 5 days, and most preferably at least 7 days. Of course, it should be recognized that numerous other collection modalities are also deemed appropriate, and that the cell free RNA can be at least partially purified or adsorbed to a solid phase to so increase stability prior to further processing.

As will be readily appreciated, fractionation of plasma and extraction of cell free DNA/RNA can be done in numerous manners. In one exemplary preferred aspect, whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). Cell free RNA is extracted from 2 mL of plasma using Qiagen reagents. The extraction protocol was designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated cell free RNA can be frozen prior to further processing.

### Omics Data Processing

Once cell free DNA/RNA is isolated, various types of omics data can be obtained using any suitable methods. DNA sequence data will not only include the presence or absence of a gene that is associated with cancer or inflammation, but also take into account mutation data where the gene is mutated, the copy number (e.g., to identify duplication, loss of allele or heterozygosity), and epigenetic status (e.g., methylation, histone phosphorylation, nucleosome positioning, etc.). With respect to RNA sequence data it should be noted that contemplated RNA sequence data include mRNA sequence data, splice variant data, polyadenylation information, etc. Moreover, it is generally preferred that the RNA sequence data also include a metric for the transcription strength (e.g., number of transcripts of a damage repair gene per million total transcripts, number of transcripts of a damage repair gene per total number of transcripts for all damage repair genes, number of transcripts of a damage repair gene per number of transcripts for actin or other household gene RNA, etc.), and for the transcript stability (e.g., a length of poly A tail, etc.).

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

With respect to the transcription strength (expression level), transcription strength of the cell free RNA can be examined by quantifying the cell free RNA. Quantification of cell free RNA can be performed in numerous manners, however, expression of analytes is preferably measured by quantitative real-time RT-PCR of cell free RNA using primers specific for each gene. For example, amplification can be performed using an assay in a 10 μL reaction mix containing 2 μL cell free RNA, primers, and probe. mRNA of α-actin can be used as an internal control for the input level of cell free RNA. A standard curve of samples with known concentrations of each analyte was included in each PCR plate as well as positive and negative controls for each gene. Test samples were identified by scanning the 2D barcode on the matrix tubes containing the nucleic acids. Delta Ct (dCT) was calculated from the Ct value derived from quantitative PCR (qPCR) amplification for each analyte subtracted by the Ct value of actin for each individual patient's blood sample. Relative expression of patient specimens is calculated using a standard curve of delta Cts of serial dilutions of Universal Human Reference RNA set at a gene expression value of 10 (when the delta CTs were plotted against the log concentration of each analyte).

Alternatively, where discovery or scanning for new mutations or changes in expression of a particular gene is desired, real time quantitative PCR may be replaced by RNAseq to so cover at least part of a patient transcriptome. Moreover, it should be appreciated that analysis can be performed static or over a time course with repeated sampling to obtain a dynamic picture without the need for biopsy of the tumor or a metastasis. Moreover, where suitable, cfRNA can be quantified using various hybridization protocols with detectable label, quantities permitting.

Consequently, the transcriptomics data may be associated with one or more protein expression level(s) or status of one or more protein(s) in the cancer tissue. Viewed from different perspective, the transcriptomics data may be used to infer one or more protein expression level(s) or status of one or more protein(s) in the cancer tissue. For example, a specific mutation detected in a transcript of a gene may indicate loss of expression in protein level (even if quantity of transcripts are not substantially affected), or gain/loss of function of the protein. In another example, increase or decrease of RNA expression levels may indicate the over- or under-expression of the protein translated from the gene.

### Data Analysis

One-On-One Analysis:

As already noted above, it should be appreciated that where cfRNA was obtained before treatment (e.g., chemotherapy, radiation therapy, surgery), the quantities of corresponding cfRNA sequences may be compared before and after treatment. Thus, and viewed from a different perspective, the quantitative cfRNA measurements before and after treatment will typically directly correlate with the number of residual cancer cells. Where the cfRNA or portion thereof is unique to the patient (e.g., where the sequence covers RNA encoding a patient and tumor specific neoepitope), the quantitative information may be obtained with substantially no false positive background. Moreover, repeated quantification of the cfRNA may provide a trend (upwards or downwards as a function of treatment. Therefore, contemplated analyses will also be suitable for predicting treatment effects and/or likely treatment outcome.

One-on-one analyses may also include quantification of cfRNA encoding genes that were used in the therapy, and particularly cfRNA that encodes neoepitopes. Such information is significant as it may confirm at least transcription of the recombinant sequences used in the therapy, which may be indicative of the likely treatment outcome in that patient.

Still further, it should be recognized that where multiple cfRNA sequences are surveilled, treatment may be followed in a statistically more significant manner. Additionally, it should be appreciated that multiple cfRNA sequences may also provide an indication of clonal shift within a tumor cell population. For example, while one set of neoepitope sequences may diminish, other neoepitope sequences may persist or even increase, thereby indicating treatment resistance or emergence of a new clonal population.

Known tumor sequence analyses: Similar to the above, where the cfRNA encode tumor associated or tumor specific genes, quantitative analysis of these cfRNA sequences may provide real-time information of residual tumor cells independent of patient specific neoepitopes. Thus, in such method off the shelf test systems can be immediately deployed. Moreover, where data for other patients are available for which the same sequences were monitored, dynamic changes can be followed and attributed to one or more known outcomes (e.g., slow decline over 6 weeks of cfRNA encoding PSA may be indicative of radiation success). Likewise, plateauing or decline to a specific value may be indicative of eradication of the tumor and residual quantities may be due to background signal. Known cfRNA sequences may also include a representative panel of genes that are known to be affected by the therapy. As such, quantification of cfRNA of such genes may provide a more systemic picture of treatment success and/or minimal residual disease.

Identification of Patterns:

In view of the above and the universal detectability of cfRNA in blood, it should be noted that where quantities of multiple cfRNA sequences are measured, numerous patterns may be established. For example, contemplated patterns may be tumor specific (as will be in the case of tumor related cfRNA sequences) or may be reflective of systemic events, including DNA repair status, inflammation status, EMT status, and checkpoint inhibition status. Most notably, such systemic status indications may further provide detail information suitable for prognosis or change in treatment. For example, where the cfRNA analysis during and/or after therapy indicates an increase in checkpoint inhibition, plateauing of tumor specific cfRNA sequences may be indicative of treatment success where checkpoint inhibitors are available. On the other hand, where EMT markers are upregulated, treatment may be adopted to reduce TNF-alpha or IL-8.

Likewise, and especially where quantities of multiple cfRNA transcripts that are unique to or associated with the tumor are measured, patterns may be established, both along a temporal and a quantitative axis. For example, increased quantity (expression level) of gene A transcript (e.g., of at least 20%, at least 30%, at least 50%, etc.) is associated with increased quantity (expression level) of gene B transcript (e.g., of at least 20%, at least 30%, at least 50%, etc.) for at least 60%, at least 70%, at least 80% of samples, the pattern can be established that co-increased expression of gene A and gene B transcripts may be associated with the prognosis of minimal residual disease. Viewed from different perspective, where the increased quantity of gene A transcript is detected in a sample, such observation may trigger or encourage the next analysis of quantification of gene B transcript to confirm the status of the tissue (e.g., associated with minimal residual disease, etc.). The inventors contemplate that the patterns may include co-increased expression (independently or dependently), co-decreased expression (independently or dependently), and/or inversed expressions of two or more genes (one increased and another decreased, including sliding scale-type relationship). Such patterns will advantageously provide information about the speed or dynamic of treatment response, as well as emergence of resistant cells or clones.

Pathway Analysis:

In some embodiments, the transcriptomics data of one or more genes can be used as an input into pathway analysis algorithms to identify affected and/or targetable pathways and/or intrinsic properties of the tumor tissue or cells. In some embodiments, the transcriptomics data of selected genes (in each cluster or one of the clusters) can be integrated into a pathway model (e.g., as a pathway element or a regulatory parameter to control or affect the pathway element, etc.) to generate a modified pathway of cancer tissue to determine any differential pathway characteristic of the cancer tissue. While any suitable methods of analyzing pathway characteristics of cells are contemplated, a preferred method uses PARADIGM (Pathway Recognition Algorithm using Data Integration on Genomic Models), which is a genomic analysis tool described in WO2011/139345 and WO/2013/062505 and uses a probabilistic graphical model to integrate multiple genomic data types on curated pathway databases.

Additional Analyses:

The above analyses may also be combined with the general error status for an individual (or tumor within an individual), or with the number and/or type of alterations in cancer-related genes, inflammation-related genes, or a DNA-repair gene to identify a ‘tipping point’ for one or more gene mutations after which a general mutation rate skyrockets. Such early warning system is particularly beneficial to avoid establishment of a new clonal population that may be more difficult to treat once established. For example, where a rate or number of mutations in ERCC1 and other DNA repair genes could have only minor systemic consequence, addition of further mutations to TP53 may result in a catastrophic increase in mutation rates.

Of course, it should be appreciated that analyses presented herein may be performed over specific and diverse populations to so obtain reference values for the specific populations, such as across various treatment response states (e.g., remission, partial remission, recurring disease, treatment resistant cells, etc.), a specific age or age bracket, a specific ethnic group that may or may not be associated with a particular responsiveness to a specific type of treatment. Of course, populations may also be enlisted from databases with known omics information, and especially publically available omics information from cancer patients (e.g., TCGA, COSMIC, etc.) and proprietary databases from a large variety of individuals that may be healthy or diagnosed with a disease. Likewise, it should be appreciated that the population records may also be indexed over time for the same individual or group of individuals, which advantageously allows detection of shifts or changes in the genes and pathways associated with different types of cancers.

In further particularly preferred aspects, it is contemplated that a cancer score can be established for one or more cancer-related genes, inflammation-related genes, a DNA-repair gene, a neoepitope, and a gene not associated with a disease and that the score may be reflective of or even prognostic for various types of cancer that are at least in part due to mutations in cancer-related genes and/or pathways. For example, especially suitable cancer scores may involve scores for one or more genes associated with one or more types of cancer (e.g., BRCA1, BRCA2, P53, etc.) relative to another gene that may or may not be associated with one type of cancer (e.g., housekeeping genes, etc.). In another example, contemplated cancer scores may involve scores for one or more genes associated with one or more types of one or more types of cancer (e.g., BRCA1, BRCA2, P53, etc.) relative to an overall mutation rate (e.g., mutation rate of the genes not associated with a disease, etc.) to so better identify cancer relevant mutations over ‘background’ mutations. Such scores can then be combined with the above analyses to further refine results and/or predicted treatment outcomes. It is also contemplated that the patient's cancer score can be compared with one or more other patients having same type of cancer and having a treatment history to provide a treatment option and predicted outcome.

Further, it is also contemplated that the transcriptomics data and/or analysis data using such transcriptomics data may be advantageously associated (preferably via machine learning) with a desired treatment or predictive parameter. For example, the transcriptomics data and/or analysis data may indicate the effect of tumor treatment. For example, where an acute lymphoblastic leukemia patient has been treated with chemotherapy using drug C for 8 weeks, and the transcriptomics data on genes A and B that are highly related to the prognosis of minimal residual diseases show the development and/or progress of minimal residual diseases (e.g., by increased RNA expression of both genes A and B, etc.), such transcriptomics data not only suggests the presence of minimal residual disease in the patient, but also implicates that chemotherapy using drug C has not been sufficiently effective to eliminate tumor cells from the patient.

Consequently, the inventors further contemplate that transcriptomics data and/or analysis data using such transcriptomics data may be used to predict likelihood of success of a treatment in treating the minimal residual disease to so generate and/or determine a treatment regimen for the patient. For example, where the transcriptomics data and/or analysis data indicates that the chemotherapy using drug C has not been sufficiently effective to eliminate tumor cells from the patient, the treatment regimen can be generated to include other types of tumor treatments (e.g., radiotherapy, stem cell transplant, etc.). Alternatively and/or additionally, the treatment regimen may include another drug(s) that has high (or at least better) likelihood of success to treat the remained tumor cells than drug C. Typically, the likelihood of success may be determined by empirical or clinical data (e.g., treatment data of other similar patients), patient's own treatment history, and/or pathway analysis (e.g., a drug targeting a gene (or a protein encoded by the gene) that shows abnormally high activity in the pathway analysis, etc.). As used here, a treatment targeting a gene refers a treatment targeting (e.g., binding, inhibiting the activity, enhancing the activity, etc.) a protein encoded by the gene, and/or a treatment inhibiting or enhancing the gene expression of the one or more genes in a transcriptional level, in a translational level, and/or in a post-translational modification level (e.g., phosphorylation, glycosylation, protein-protein binding, etc.). For example, where the transcriptomics data and/or pathway analysis data using the transcriptomics data indicates overexpression of a kinase or abnormally high kinase activity, the treatment regimen may include a kinase inhibitor. In another example, where the transcriptomics data and/or pathway analysis data using the transcriptomics data indicates expression of a neoepitope on a tumor cell (e.g., detection of a gene mutation that is likely to be expressed as a neoepitope loaded in the WIC complex, etc.), the treatment regimen may include an immune therapy (e.g., viral vaccine, bacterial vaccine, yeast vaccine including a recombinant nucleic acid encoding the neoepitope, etc.). In still another example, where the transcriptomics data and/or pathway analysis data using the transcriptomics data indicates a dominant negative protein-protein interaction (e.g., abnormal binding of protein A to protein B to inhibit protein B's activity, etc.), the treatment regimen may include a binding motif to protein A to reduce the binding affinity of protein A to protein B.

Such determined or generated treatment (regimen) can be further administered to the patient diagnosed with minimal residual disease in a dose and a schedule effective or sufficient to treat the tumor (e.g., to reduce the number of remained tumor cells in the blood stream, to reduce the size of the tumor, to increase the immune response against the tumor, to increase the survival rate, etc.). As used herein, the term “administering” refers to both direct and indirect administration of the treatment regimens, drugs, therapies contemplated herein, where direct administration is typically performed by a health care professional (e.g., physician, nurse, etc.), while indirect administration typically includes a step of providing or making the compounds and compositions available to the health care professional for direct administration.

Further, the transcriptomics data and/or analysis data using such transcriptomics data may be used to predict the survival time, overall survival rate or a disease free or progression free survival time.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A method of determining presence of minimal residual disease in a patient, the method comprising:
obtaining cfRNA from blood of the patient;
identifying sequence information that is specific for at least one expressed gene in a tumor of the patient, wherein the step of obtaining and and the step of identifying are each performed before treatment of the patient;
obtaining, after treatment of the patient, cfRNA from blood of the patient; and
using the cfRNA to quantify the at least one expressed gene.

2. The method of claim 1, wherein the step of obtaining sequence information comprises data transfer of sequence data from a database, and/or wherein the step of identifying sequence information comprises omics analysis of the tumor.

3-9. (canceled)

10. The method of claim 1, wherein the at least one expressed gene is at least one of a cancer-related gene, a cancer-specific gene, a DNA-repair gene, a checkpoint related gene, and a gene comprising a sequence encoding a patient- and tumor specific neoepitope.

11. The method of claim 10, wherein the sequence information is specific for at least ten expressed genes in a tumor of the patient.

12. The method of claim 1, wherein the treatment of the patient is at least one of chemotherapy, radiation therapy, and surgery.

13. The method of claim 1, wherein the cfRNA is substantially devoid of DNA.

14. The method of claim 1, wherein the at least one expressed gene is quantified using OCR.

15. The method of claim 1, further comprising a step of identifying a signature of the at least one expressed gene.

16. The method of claim 1, further comprising a step of correlating the at least one expressed gene with a response to the treatment.

17. A method of determining presence of minimal residual disease in a patient, the method comprising:
identifying, after treatment of the patient, at least two expressed gene of a treated tumor from cfRNA of the patient, wherein the cfRNA is obtained from blood of the patient; and
correlating presence of minimal residual disease with a threshold quantity and/or pattern of the at least two expressed genes.

18. The method of claim 17, wherein the step of identifying the at least two expressed genes further comprises a step of quantifying the cfRNA for the at least two expressed, genes.

19-26. (canceled)

27. The method of claim 17, wherein the at least two expressed genes are selected from the group consisting of a cancer-related gene, a cancer-specific gene, a DNA-repair gene, a checkpoint related gene, and a gene comprising a sequence encoding a patient- and tumor-specific neoepitope.

28. (canceled)

29. The method of claim 17, wherein the treatment of the patient is at least one of chemotherapy, radiation therapy, and surgery.

30. The method of claim 17, wherein the cfRNA is substantially devoid of DNA.

31. The method of claim 17, wherein the threshold quantity is a detection limit for qPCR.

32. The method of claim 17, wherein the threshold quantity is at least 20% of a measured quantity of at least one of the at least two expressed genes before treatment.

33. The method of claim 17, wherein the pattern is a pattern that is characteristic for recurring disease, treatment resistance, and/or immune suppression.

34. The method of claim 33, wherein the pattern is a pattern from a different patient.

35-42. (canceled)
